SEQUENCE LISTING (26 sequences)

SEQ ID NO: 1 - DNA, 854 bp, Artificial Sequence
  NME7 GENBANK ACCESSION AB209049
gagatcctgagacaatgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggtatgttgaatacactatattcagtacattttgttaataggagagcaatgtttattttcttgatgtactttatgtatagaaaataa

SEQ ID NO: 2 - protein, 283 aa, Artificial Sequence
  NME7 GENBANK ACCESSION AB209049
DPETMNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGMLNTLYSVHFVNRRAMFIFLMYFMYRK

SEQ ID NO: 3 - DNA, 534 bp, Artificial Sequence
  NM23-H1 GENBANK ACCESSION AF487339
atggtgctactgtctactttagggatcgtctttcaaggcgaggggcctcctatctcaagctgtgatacaggaaccatggccaactgtgagcgtaccttcattgcgatcaaaccagatggggtccagcggggtcttgtgggagagattatcaagcgttttgagcagaaaggattccgccttgttggtctgaaattcatgcaagcttccgaagatcttctcaaggaacactacgttgacctgaaggaccgtccattctttgccggcctggtgaaatacatgcactcagggccggtagttgccatggtctgggaggggctgaatgtggtgaagacgggccgagtcatgctcggggagaccaaccctgcagactccaagcctgggaccatccgtggagacttctgcatacaagttggcaggaacattatacatggcagtgattctgtggagagtgcagagaaggagatcggcttgtggtttcaccctgaggaactggtagattacacgagctgtgctcagaactggatctatgaatga

SEQ ID NO: 4 - protein, 177 aa, Artificial Sequence
  NM23-H1 GENBANK ACCESSION AF487339
MVLLSTLGIVFQGEGPPISSCDTGTMANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGSDSVESAEKEIGLWFHPEELVDYTSCAQNWIYE

SEQ ID NO: 5 - DNA, 399 bp, Artificial Sequence
  Human NME7-A
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgtttttttga

SEQ ID NO: 6 - protein, 132 aa, Artificial Sequence
  Human NME7-A
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFF

SEQ ID NO: 7 - DNA, 444 bp, Artificial Sequence
  Human NME7-A1
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttacttga

SEQ ID NO: 8 - protein, 147 aa, Artificial Sequence
  Human NME7-A1
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFT

SEQ ID NO: 9 - DNA, 669 bp, Artificial Sequence
  Human NME7-A2
atgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgtttttttga

SEQ ID NO: 10 - protein, 222 aa, Artificial Sequence
  Human NME7-A2
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFF

SEQ ID NO: 11 - DNA, 714 bp, Artificial Sequence
  Human NME7-A3
atgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttacttga

SEQ ID NO: 12 - protein, 237 aa, Artificial Sequence
  Human NME7-A3
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFT

SEQ ID NO: 13 - DNA, 408 bp, Artificial Sequence
  Human NME7-B
atgaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttctga

SEQ ID NO: 14 - protein, 135 aa, Artificial Sequence
  Human NME7-B
MNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 15 - DNA, 426 bp, Artificial Sequence
  Human NME7-B1
atgaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattagtga

SEQ ID NO: 16 - protein, 140 aa, Artificial Sequence
  Human NME7-B1
MNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 17 - DNA, 453 bp, Artificial Sequence
  Human NME7-B2
atgccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttctga

SEQ ID NO: 18 - protein, 150 aa, Artificial Sequence
  Human NME7-B2
MPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 19 - DNA, 471 bp, Artificial Sequence
  Human NME7-B3
atgccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattagtga

SEQ ID NO: 20 - protein, 155 aa, Artificial Sequence
  Human NME7-B3
MPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 21 - DNA, 864 bp, Artificial Sequence
  Human NME7-AB
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattagtga

SEQ ID NO: 22 - protein, 286 aa, Artificial Sequence
  Human NME7-AB
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 23 - DNA, 846 bp, Artificial Sequence
  Human NME7-AB1
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttctga

SEQ ID NO: 24 - protein, 281 aa, Artificial Sequence
  Human NME7-AB1
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 25 - DNA, 759 bp, Artificial Sequence
  Human NME7-X1
atgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattag

SEQ ID NO: 26 - protein, 252 aa, Artificial Sequence [sequence omitted]

(19) United States
(12) Patent Application Publication
(10) Pub. No.: US 2021/0308213 A1
(43) Pub. Date: Oct. 7, 2021

(54) METHOD FOR DIAGNOSING AND TREATING CANCER USING NAÏVE STATE STEM CELL SPECIFIC GENES

(71) Applicant: MINERVA BIOTECHNOLOGIES CORPORATION, Waltham, MA (US)

(72) Inventors: Cynthia Bamdad, Waltham, MA (US); Benoit Smagghe, Waltham, MA (US); Mark G. Carter, Waltham, MA (US)

(21) Appl. No.: 17/353,704

(22) Filed: Jun. 21, 2021

Related U.S. Application Data
(63) Continuation of application No. 15/060,484, filed on Mar. 3, 2016.
(60) Provisional application No. 62/127,746, filed on Mar. 3, 2015.

(51) Int. Cl. A61K 38/17; C12Q 1/6886

(52) U.S. Cl. CPC A61K 38/17; C12Q 2600/158; C12Q 1/6886

(57) ABSTRACT

The present application discloses a method for converting a cell to naïve state stem cells comprising contacting the cell to be converted with an NME protein.

### CROSS-REFERENCE TO RELATED APPLICATIONS

The present application is a continuation of U.S. patent application Ser. No. 15/060,484, filed Mar. 3, 2016, which claims the benefit of priority to U.S. Provisional Patent Application No. 62/127,746, filed Mar. 3, 2015, each of which hereby is incorporated by reference in their entirety.

### BACKGROUND OF THE INVENTION

### 1. Field of the Invention

The present application relates to methods of treating cancer.

### 2. General Background and State of the Art

In order to effectively treat cancer, it is important to develop drugs that target the molecules that drive the growth and metastasis of cancers. The recent development of deep sequencing technologies has now made it possible to compare the expression level of thousands of genes in a first cell population to expression levels of those same genes in a second cell population. One method of discovering new cancer drug targets is to identify genes that are specifically up- or down-regulated in cancer cells compared to healthy cells. However, this method combined with person-to-person variation among cancers, has generated very long lists of potential cancer drug target genes, with no way of identifying those genes that drive cancer rather than those that are artifacts of cancer. Therefore, what is needed is a method for identifying those few genes that are critical to the progression of cancer, so that drugs that disable them can be developed. Because metastatic cancer is what kills the patient and there is no effective treatment for metastatic cancer, what would be an improvement would be a method for identifying those genes that are drivers of metastasis.

### SUMMARY OF THE INVENTION

In one aspect, the invention is directed to a method for converting a cell to naïve state stem cells comprising contacting the cell to be converted with an NME protein. The cell to be converted is primed state stem cell or somatic cell. The NME protein may be NME7, NME7AB, or NME-X1.

In another aspect, the invention is directed to a method for maintaining a naïve state stem cell to be in the naïve state, comprising contacting the cell to be converted with an NME protein.

In yet another aspect, the invention is directed to a method for determining whether a cell to be tested is naïve state stem cell, comprising comparing transcriptome signature of NME induced naïve state stem cell with the transcriptome of the cell that is tested, wherein a match in transcriptome signature indicates that the tested cell is naïve state stem cell. The NME induced naïve state stem cell transcriptome signature may be represented in Tables 1, 2, 3, and 5.

In yet another aspect, the invention is directed to a method for determining whether a cell to be tested is naïve state stem cell, comprising comparing whether any of about 3 to 17, 3 to 15, 2 to 14, 4 to 12, 3 to 10, 5 to 8 or 3 to 6 genes of transcriptome signature of NME induced naïve state stem cell, whether expressed or inhibited, are also expressed or inhibited in the cell to be tested, wherein a match in the expression or inhibition of the genes indicates that the tested cell is naïve state stem cell. The NME induced naïve state stem cell transcriptome signature may be represented in Tables 1, 2, 3, and 5.

In yet another aspect, the invention is directed to a method for determining whether a cancer cell to be tested is metastatic cancer, comprising comparing transcriptome signature of NME induced naïve state stem cell with the transcriptome of the cell that is tested, wherein a match in transcriptome signature indicates that the tested cell is metastatic cancer cell. The NME induced naïve state stem cell transcriptome signature may be represented in Tables 1, 2, 3, and 5.

In yet another aspect, the invention is directed to a method for determining whether a cancer cell to be tested is metastatic cancer, comprising comparing whether any of about 3 to 17, 3 to 15, 2 to 14, 4 to 12, 3 to 10, 5 to 8 or 3 to 6 genes of transcriptome signature of NME induced naïve state stem cell, whether expressed or inhibited, are also expressed or inhibited in the cell to be tested, wherein a match in the expression or inhibition of the genes indicates that the tested cell is metastatic cancer cell. The NME induced naïve state stem cell transcriptome signature may be represented in Tables 1, 2, 3, and 5.

In yet another aspect, the invention is directed to a method of treating cancer comprising comparing whether any of about 3 to 17, 3 to 15, 2 to 14, 4 to 12, 3 to 10, 5 to 8 or 3 to 6 genes of transcriptome signature of NME induced naïve state stem cell, whether expressed or inhibited, are also expressed or inhibited in the cancer cell, identifying gene that is expressed or inhibited and turning the gene on or off or treating with the gene product rather than turning the gene on. If turning gene off is desired, the method includes disrupting the Super Mediator complex that super enhances the gene. If turning gene on is desired, the method includes inducing Superenhancer complex that super enhances the gene to bind to the gene. And where turning on the gene is desired, the gene may be HESS, GNAS, FBXL17, RHOC, VLDLR, GREB1L, EXT1, BRD2 and CDH9. The NME induced naïve state stem cell transcriptome signature may be represented in Tables 1, 2, 3, and 5. And if turning gene on is desired, the proteins themselves may be administered to the patient.

In another aspect, the invention is directed to a method of treating prostate cancer comprising determining whether a prostate cell expresses NME-X1, wherein if the cell over-expresses NME-X1, then treating cells with anti-prostate cancer agents.

In yet another aspect, the invention is directed to a method of treating cancer comprising turning on any of the genes or any combination thereof in Table 5.

In yet another aspect, the invention is directed to a method of changing a cancer cell to normal cell comprising turning on any of the genes or any combination thereof in Table 5. The change in cancer cell to normal cell may occur within a patient, wherein the method includes administering to the patient a compound or a composition that turns on any of the genes or any combination thereof in Table 5.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIGS. 1A-1E show that human embryonic stem cells cultured in NME7_{AB} minimal media are pluripotent and can form teratomas. A) shows an FPLC trace and Coomassie Blue staining of reducing and non-reducing gels shows that NME7_{AB} is a 33 kDa monomeric protein. B) shows photos of NME7_{AB}-grown hESCs on anti-MUC1* antibody-coated surface show typical stem cell morphology but grow in sheets rather than in colonies. Scale bar=1 mm, 400 μm. C) shows images of immunostaining of NME7_{AB}-grown hESCs shows typical pluripotency markers. Scale bar=400 μm. D) shows photos of hematoxylin and eosin staining of teratoma sections derived from hESCs cultured in NME7_{AB} for 14 passages differentiate down all three germlines. Scale bar=100 μm. E) shows images of immunofluorescence staining for H3K27me3 foci was absent in NME7_{AB}-grown hESCs (upper) showing that both X chromosomes are active ‘XaXa’ but present in the parent FGF-grown primed state cells (lower) showing that one X has been inactivated ‘XaXi’. Scale bar=200 μm.

FIGS. 2A-2C show graphical representations of gene expression measurements using RNA-seq analysis, which show that NME7_{AB-XaXa} grown human embryonic stem cells (hESCs) are genetically the most diverse from the parent FGF_{XaXi} grown hESCs. A) Heat map of 2-way hierarchical clustering shows that the gene expression profiles of NME7_{AB-XaXa} cells, NME7_{AB-XaXi} cells, and NME1_{XaXa} cells are closely related but are very different from that of the parent FGF_{XaXi} cells. B) Principal Component Analysis (PCA) shows that the largest variance among the gene expression data sets is between FGF2 cultured stem cells and all NME cultured cells, regardless of their X-activation state. C) PCA of just the NME data sets shows that, along dimension 1, there is a clear difference in gene expression between NME7_{AB} grown stem cells, depending on their X-activation state.

FIG. 3 is a graph of RT-PCR measurements of gene expression for stem cell markers and cancer stem cell markers for T47D cancer cells after being cultured in traditional media or a media containing NME7, wherein cells that became non-adherent (floaters) were analyzed separate from those that remained adherent.

FIG. 4 is a graph of RT-PCR measurements of gene expression for a variety of stem and putative cancer stem cell markers for DU145 prostate cancer cells. Cells were cultured either in traditional media or a media containing NME1 dimers (“NM23”) or NME7 (NME7-AB). Rho kinase inhibitor was not used because by passage 2, cells remained adherent.

FIG. 5 shows photographs of two female athymice nu/nu mice out of 24 that were xenografted with only 50 human breast cancer cells that had first been grown for 7 days in NME7-AB and showed greatly increased expression of CXCR4, CHD1 and stem cell markers. In addition, half the mice were also injected daily with human recombinant NME7-AB. 82% of the mice that were also injected daily with NME7-AB developed remote metastases as well as tumors at the site of injection.

FIG. 6 shows a table of the results of the experiment in which mice were xenografted with cancer cells that were transformed to a more metastatic state by pre-culture in a medium containing human NME7-AB.

FIG. 7 shows a graph of tumor volume measurements for four (4) groups of immune-compromised nu/nu female mice implanted with either 50, 100, 1,000 or 10,000 cells subcutaneously in the flank wherein the cells that were implanted were human MUC1-positive breast cancer cells that were cultured for seven (7) days in recombinant human NME7-AB wherein the ‘floaters’ were collected and verified to overexpress metastasis receptor CXCR4 by more than 100-fold. Half the mice in each group were injected daily with human recombinant NME7-AB. Numbers within the graph refer to the mouse tracking number. ‘M’ denotes a mouse with multiple tumors.

FIG. 8 shows a graph of tumor volume measurements for four (4) groups of immune-compromised nu/nu female mice implanted with either 50, 100, 1,000 or 10,000 cells subcutaneously in the flank wherein the cells that were implanted were human MUC1-positive breast cancer cells that were cultured for seven (7) days in recombinant human NME7-AB wherein the ‘floaters’ were collected and verified to overexpress metastasis receptor CXCR4 by more than 100-fold. Half the mice in each group were injected daily with human recombinant NME7-AB. Of the mice that received daily injections of NME7-AB, 80% developed multiple tumors. This graph shows the combined volumes of multiple tumors in the same mouse. Numbers within the graph refer to the mouse tracking number. ‘M’ denotes a mouse with multiple tumors.

FIGS. 9A-9G show photographs of Western blots of a co-immunoprecipitation experiment. T47D breast cancer cell extracts were incubated with an antibody against the MUC1 cytoplasmic tail, Ab-5, or a control antibody, IgG, and co-immunoprecipitated. The gels were blotted with two different commercially available anti-NME7 antibodies B9 (A) and CF7 (B). Both gels show unique NME7 bands at ˜33 kDa and ˜30 kDa. The gels were stripped and re-probed with an antibody against the extracellular domain of MUC1*, anti-PSMGFR (C) and (D), which shows that the NME7 species and MUC1* interact. A recombinant NME7-AB and a recombinant NME7-X1 were mixed together and run on a gel, then probed with an anti-NME7 antibody, showing that the two unique NME7 species that are naturally occurring in breast cancer cells and that interact with MUC1* are an NME7-AB-like species and NME7-X1 (E). Western blots of a co-immunoprecipitation experiment. Human induced pluripotent stem, iPS7, or embryonic stem, HES3, cell extracts were incubated with an antibody against the MUC1 cytoplasmic tail, Ab-5, or a control antibody, IgG, and co-immunoprecipitated. The gel was blotted with a commercially available anti-NME7 antibody B9 (F). Both cell types show unique NME7 bands at ˜33 kDa and ˜30 kDa. The gel was stripped and re-probed with an antibody against the extracellular domain of MUC1*, anti-PSMGFR (G), which shows that the NME7 species and MUC1* interact.

FIG. 9H shows a graph of RT-PCR measurement of the expression of NME7-X1 in a panel of human stem cells and cancer cells.

FIG. 9I shows sequence alignment of NME7-A, also know as variant 1 or v1, NME7-B, also know as variant 2 or v2, and NME7-X1, also know as X1. Primers that enable detection of NME7-X1 specifically and differentiated from NME7 are indicated.

FIG. 10 is Table 1, which shows measured differences in gene expression, 2-fold or greater, between primed state stem cells and naïve state stem cells using RNA-seq. Column D) human embryonic stem cells (hESCs) that had been cultured in standard FGF2 and were verified to be 100% primed by virtue of inactivation of second X chromosome, were cultured in NME7-AB and verified to be 100% naïve by virtue of re-activation of second X chromosome (Carter M G, Smagghe B J, Stewart A K et al. A Primitive Growth Factor, NME7_{AB}, Is Sufficient to Induce Stable Naïve State Human Pluripotency; Reprogramming in This Novel Growth Factor Confers Superior Differentiation. Stem Cells. 2016: DOI: 10.1002/stem.2261. Column E) hESCs and induced pluripotent stem (iPS) cells reverted to a naïve-like state by culture in FGF2, LIF and a cocktail of biochemical inhibitors (extracted from Theunissen T W, Powell B E, Wang H et al. Systematic identification of culture conditions for induction and maintenance of naive human pluripotency. Cell Stem Cell. 2014; 15:471-487.) F) hESCs and induced pluripotent stem (iPS) cells reverted to a naïve-like state by culture in FGF2, LIF and a cocktail of biochemical inhibitors (extracted from Gafni O, Weinberger L, Mansour A A et al. Derivation of novel human ground state naive pluripotent stem cells. Nature. 2013; 504:282-286.)

FIG. 11 is Table 2, which lists genes that only had altered expression in NME7-AB induced naïve state stem cells, compared to the primed state parent cells.

FIG. 12 is Table 3, which lists the genes that had altered expression in all three sets of naïve stem cells, Carter et al, Theunissen et al, or Gafni et al, regardless of the method for reverting them to the earlier naïve state.

FIG. 13 is Table 4, which lists genes that are occupied by superenhancers in human embryonic stem cells H1s, which are in the primed state (extracted from Hnisz et al.)

FIG. 14 is Table 5, which lists genes that Hnisz et report are occupied by Superenhancers in primed state human stem cells and are superexpressed, but that we discovered are under expressed in naïve stem cells.

FIG. 15 is Table 6, which shows nucleic acid primers that we designed which are able to distinguish NME7 from an alternative isoform we discovered called NME-X1.

FIG. 16 is Table 7, which shows nucleic acid sequences that are able to detect NME7-X1 and NME7 and able to distinguish one from the other via hybridization.

### DETAILED DESCRIPTION OF THE PREFERRED EMBODIMENTS

In the present application, “a” and “an” are used to refer to both single and a plurality of objects.

As used herein “sequence identity” means homology in sequence of a particular polypeptide or nucleic acid to a reference sequence of nucleic acid or amino acid such that the function of the homologous peptide is the same as the reference peptide or nucleic acid. Such homology can be so close with the reference peptide such that at times the two sequences may be 90%, 95% or 98% identical yet possess the same function in binding or other biological activities.

As used herein, “transcriptome” refers to the full range of mRNA molecules expressed by an organism, particular tissue type or cell type. In this regard, “transcriptome signature of NME induced naïve state stem cell” refers to the full range of mRNA molecules expressed in naïve state stem cells induced by NME.

As used herein, “NME family proteins” or “NME family member proteins”, numbered 1-10, are proteins grouped together because they all have at least one NDPK (nucleotide diphosphate kinase) domain. In some cases, the NDPK domain is not functional in terms of being able to catalyze the conversion of ATP to ADP. NME proteins were formerly known as NM23 proteins, numbered H1, H2 and so on. Herein, the terms NM23 and NME are interchangeable. Herein, terms NME1, NME2, NME6 and NME7 are used to refer to the native protein as well as NME variants. In some cases these variants are more soluble, express better in E. coli or are more soluble than the native sequence protein. For example, NME7 as used in the specification can mean the native protein or a variant, such as NME7-AB that has superior commercial applicability because variations allow high yield expression of the soluble, properly folded protein in E. coli. “NME1” as referred to herein is interchangeable with “NM23-H1”. It is also intended that the invention not be limited by the exact sequence of the NME proteins. NME7 as referred to herein is intended to mean native NME7 having a molecular weight of about 42 kDa, a cleaved form having a molecular weight between 25 and 33 kDa, a variant devoid of the DM10 leader sequence, NME7-AB or a recombinant NME7 protein, or variants thereof whose sequence may be altered to allow for efficient expression or that increase yield, solubility or other characteristics that make the NME7 more effective or commercially more viable.

As used herein, an “an agent that maintains stem cells in the naïve state or reverts primed stem cells to the naïve state” refers to a protein, small molecule or nucleic acid that alone or in combination maintains stem cells in the naïve state, resembling cells of the inner cell mass of an embryo. Examples include but are not limited to NME1 dimers, human or bacterial, NME7, NME7-AB, 2i, 5i, nucleic acids such as siRNA that suppress expression of MBD3, CHD4, BRD4, or JMJD6.

As used herein, the term “cancer stem cells” or “tumor initiating cells” refers to cancer cells that express levels of genes that have been linked to a more metastatic state or more aggressive cancers. The terms “cancer stem cells” or “tumor initiating cells” can also refer to cancer cells for which far fewer cells are required to give rise to a tumor when transplanted into an animal. Cancer stem cells and tumor initiating cells are often resistant to chemotherapy drugs.

As used herein, the terms “stem/cancer”, “cancer-like”, “stem-like” refers to a state in which cells acquire characteristics of stem cells or cancer cells, share important elements of the gene expression profile of stem cells, cancer cells or cancer stem cells. Stem-like cells may be somatic cells undergoing induction to a less mature state, such as increasing expression of pluripotency genes. Stem-like cells also refers to cells that have undergone some de-differentiation or are in a meta-stable state from which they can alter their terminal differentiation. Cancer like cells may be cancer cells that have not yet been fully characterized but display morphology and characteristics of cancer cells, such as being able to grow anchorage-independently or being able to give rise to a tumor in an animal.

Cancer and Naïve Stem Cells

For some time, oncologists have observed that the more progressed a cancer is, the more de-differentiated the cancer cells look. The inventors have discovered that the more severe or metastatic the cancer is, the more the cancer cells look like stem cells, visually as well as molecularly. All pluripotent human stem cells express MUC1* (Hikita et al 2008). Similarly, over 75% of cancers express MUC1* (Mahanta et al 2008). In stem cells, the growth factor that binds to and activates the MUC1* growth factor receptor is dimeric NME1 (Smagghe 2013), NME7, or an NME7 variant, such as a 33 kDa NME7 cleavage product (Carter et al 2016). In cancer cells, the growth factor that binds to and activates the MUC1* growth factor receptor is dimeric NME1, NME7, or an NME7 variant, such as a 33 kDa NME7 cleavage product. In both stem cells and cancer cells NME1 and NME7 promote growth, pluripotency and inhibition of differentiation.

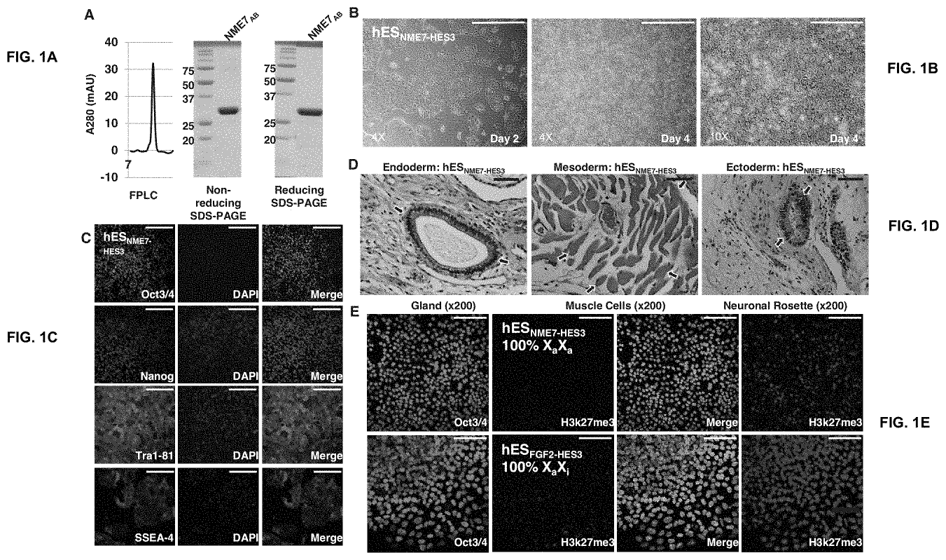

Both cancer cells and stem cells can be propagated by culturing them in NME proteins, including dimeric NME1 and monomeric NME7, particularly NME7 variants that lack or have a truncated N-terminal portion, sometimes called a DM-10 domain. We made a recombinant human NME7 that is lacking the DM-10 domain and comprises two NDPK domains called A and B. We call this 33 kDa recombinant protein “NME7_{AB}” (Carter et al, Stem Cells 2016 DOI: 10.1002/stem.2261). When stem cells are cultured in NME7_{AB} they de-differentiated even further to an earlier stem cell stage called “naïve”. A monomeric recombinant NME7_{AB} that we made (FIG. 1A) fully supported human stem cell growth in the absence of any other growth factor (FIG. 1B). As evidence of their pluripotency, they stained positive for the typical pluripotency markers and formed teratomas (FIG. 1C,D). As evidence of their naïve state, both X chromosomes were active, XaXa, in contrast to the parent FGF2 grown cells wherein one X had already been inactivated as can be seen by focal staining for tri-methylated Lysine 27 on Histone 3 (FIG. 1E).

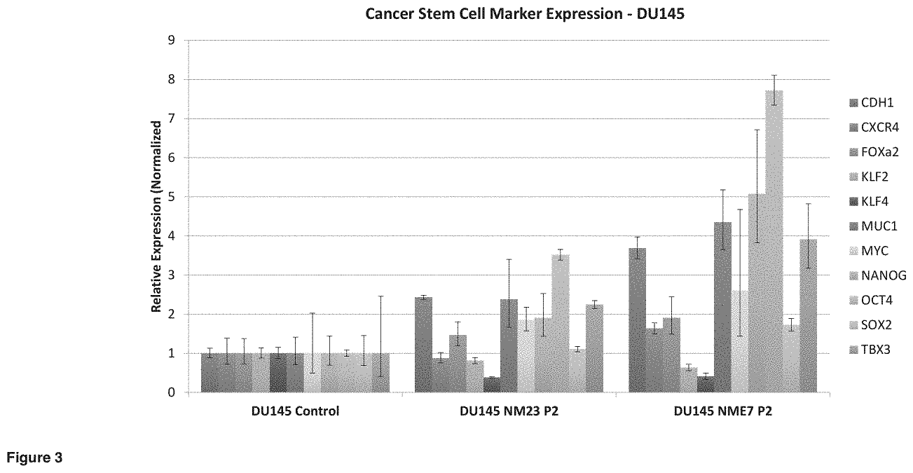

When cancer cells are grown in NME7_{AB} they de-differentiated even further and become metastatic cancer cells, which are sometimes called cancer stem cells. DU145 prostate cancer cells that were cultured in NME7-AB showed dramatic increases in expression of metastatic markers (FIG. 3). In prostate cancer cells, CHD1 (aka E-cadherin) and CXCR4 were up-regulated compared to the control cancer cells, which were not grown in NME7-AB, along with other pluripotent stem cell markers. Ovarian cancer cells, pancreatic cancer cells and melanoma cells were also cultured in NME7-AB and were transformed to a more metastatic state after as few as 3 days in culture. All transitioned from adherent to non-adherent floater cells and increased expression of metastatic markers after 72 or 144 hours in culture with NME7-AB.

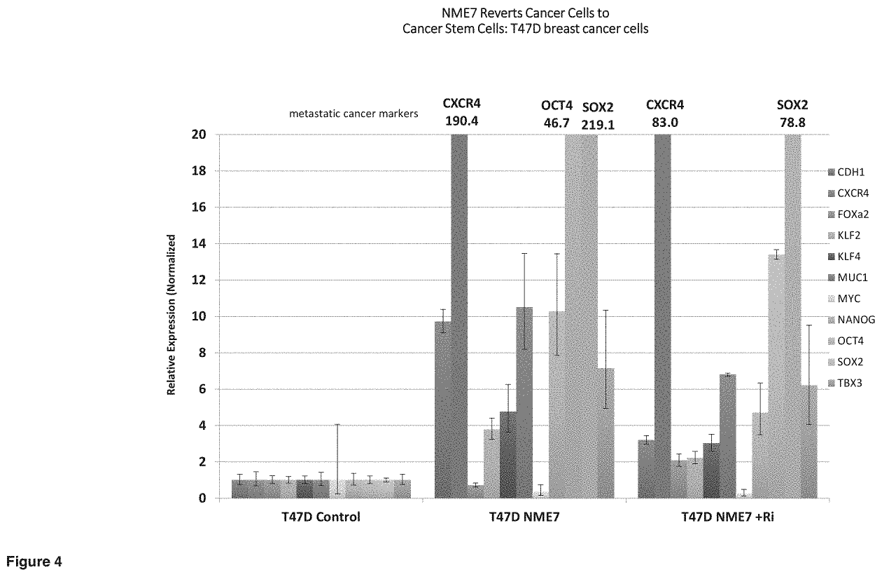

In one particular experiment, T47D human breast cancer cells were cultured in either standard RPMI media or in minimal serum-free media plus 4 nM NME7-AB. After 8 days the cells were harvested and measured by Q-PCR for the presence of metastatic markers. Compared to the control cells, NME7-AB induced dramatic increases in the expression of metastatic markers such as CXCR4, which was up-regulated by 40-200-times (FIG. 4).

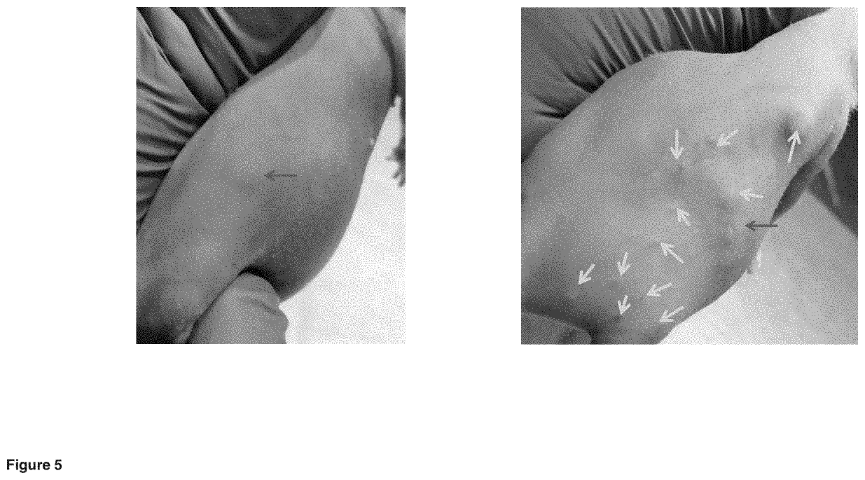

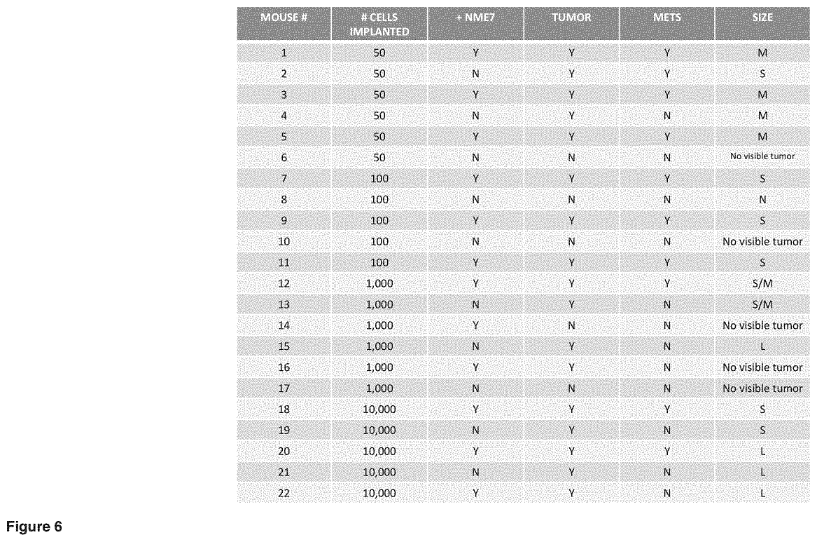

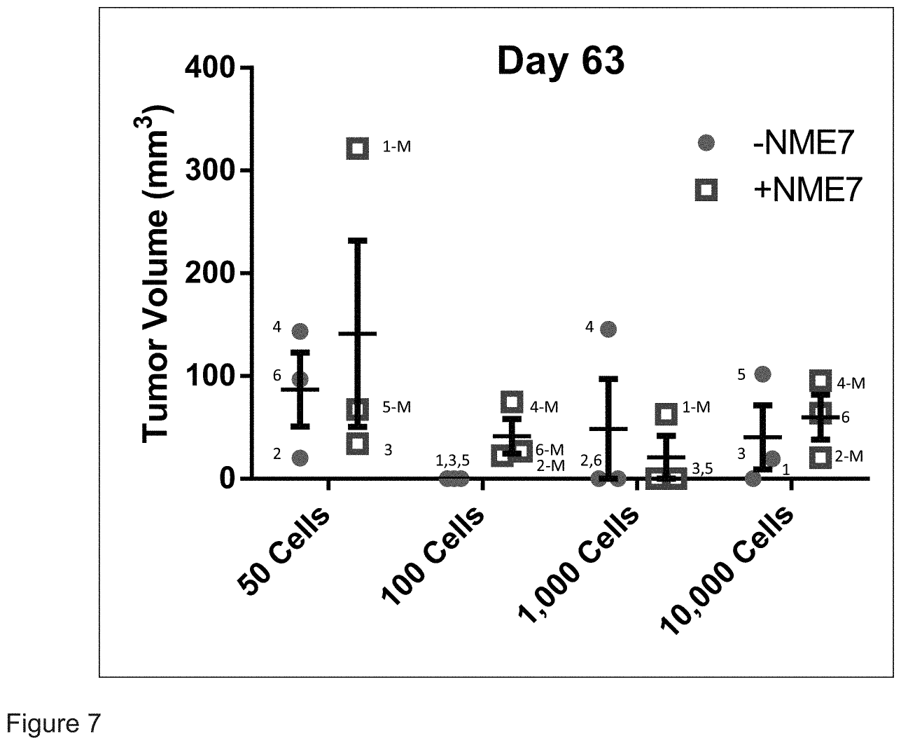

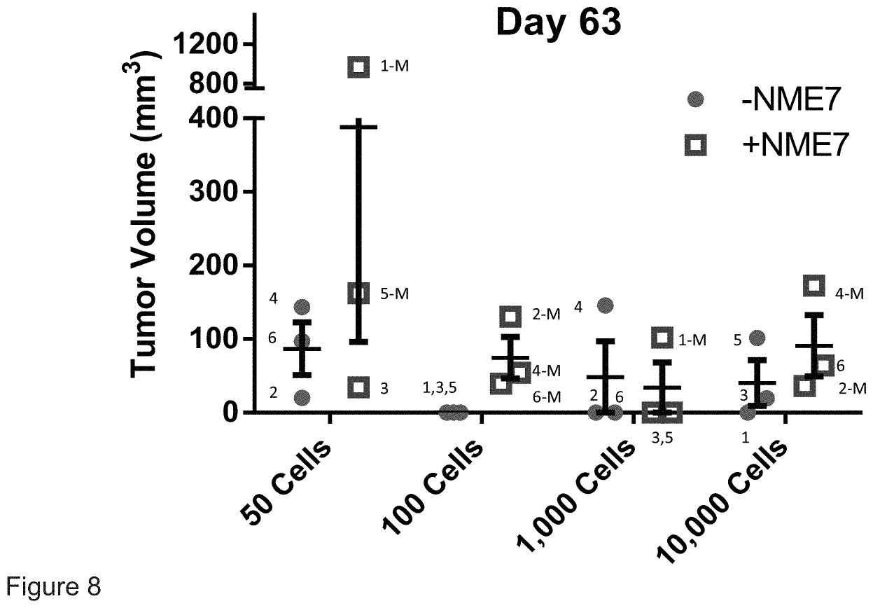

The freshly harvested NME7-AB induced metastatic cells were xenografted into the flank of female nu/nu athymic mice that have been implanted with 90-day slow release estrogen pellets. Floater cells were xenografted with 10,000, 1,000, 100 or 50 cells each. Half of the mice in each group of 6 were also injected daily with 32 nM NME7-AB near the original implantation site. The parent T47D cells that were cultured in RPMI media without NME7-AB were also implanted into mice at 6 million, 10,000 or 100 as controls. Mice implanted with the NME7-induced floater cells developed tumors even when as few as 50 cells were implanted. Mice that were implanted with the floater cells and that received daily injections of NME7-AB also developed remote tumors or remote metastases in various organs (FIG. 5). 11 out of the 12 mice, or 92%, that were injected with human NME7-AB after implantation of the NME7-AB cultured cancer cells developed tumors at the injection site. Only 7 out of the 12 mice, or 58%, that were not injected with human NME7-AB after implantation developed tumors. 9 out of the 11 mice, or 82%, that exhibited tumors and were injected with human NME7-AB developed multiple tumors remote from the injection site. None of the mice that were not injected with NME7-AB developed multiple, visible tumors (FIGS. 6-8).

Together, these data show that stem cells and cancer cells, especially metastatic cancer cells, grow by the same mechanism. NME7_{AB} drives stem cells to the earliest naïve state and NME7_{AB} drives cancer cells to the most cancerous, metastatic state. Therefore, the critical genes that drive stem cells to the naïve stem cell state are the same genes that drive cancer cells to the aggressive metastatic state. It then follows that the genes that are up- or down-regulated in naïve stem cells compared to regular stem cells are those genes that are similarly up- or down-regulated in cancers, especially in metastatic cancers. It is then concluded that genes that are differentially expressed in naïve stem cells compared to regular primed state stem cells are good targets for anti-cancer drugs. Drugs that alter the expression of one or more of those genes such that their expression levels more closely match regular primed state stem cells will be drugs to treat or prevent cancers.

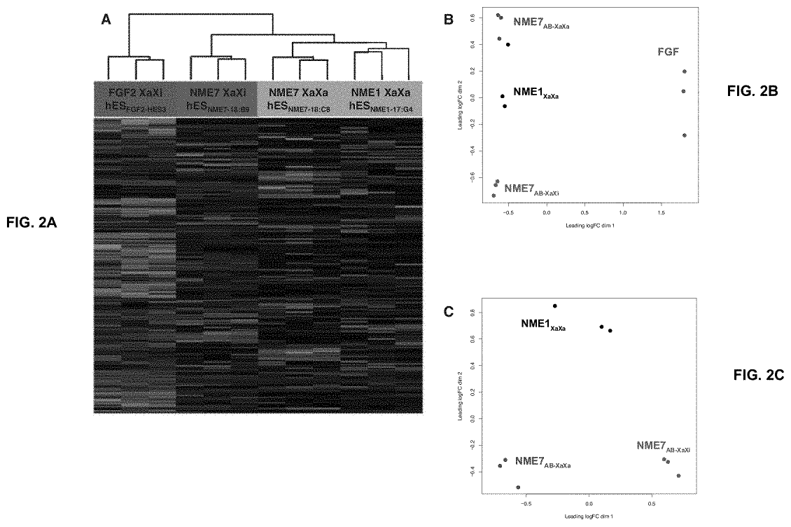

The differential gene expression signature of naïve state stem cells compared to primed stem cells identifies genes that will have altered expression in metastatic or very aggressive cancers. For example, we performed global gene expression analysis, RNA-SEQ, on human stem cells that were either in the primed state or in the naïve state. The Heat Map of FIG. 2 shows that the genetic signature of naïve state stem cells is very different from that of primed state stem cells. The cells that gave rise to both gene expression signatures are the same. The difference that caused the dramatic change in gene expression is that the stem cells were moved from the standard FGF2 media into a serum-free media containing 2-8 nM NME7-AB. We confirmed that they had been reverted to the earlier naïve state by measuring X chromosome activation and showing that the NME7_{AB}, cultured cells had re-activated the second X chromosome which is the gold standard for determining if a stem cell is in the naïve state. FIG. 2 shows a Heat Map of 2-way hierarchical clustering for human embryonic stem cells grown in FGF2 that are in the primed state, compared to the same parent cell line that was grown in NME7_{AB} and have both X chromosomes re-activated (XaXa), which verifies that they are truly naïve. Principal Component Analysis (PCA) shows that the largest variance among the gene expression data sets is between FGF2 cultured stem cells and all NME cultured cells, regardless of their X-activation state (FIG. 2B). PCA of just the NME data sets shows that, along dimension 1, there is a clear difference in gene expression between NME7_{AB} grown stem cells, depending on their X-activation state (FIG. 2C).

The precise amount that the expression of each gene changed, compared to the parent primed state cells, was measured by RNA-SEQ and is shown in Table 1; FIG. 10. Others have reverted human stem cells to a somewhat naïve state using different growth factors and biochemical inhibitors (Theunissen et al., Systematic Identification of Culture Conditions for Induction and Maintenance of Naive Human Pluripotency, Cell Stem Cell (2014), dx.doi.org/10.1016/j.stem.2014.07.002; Gafni et al., Nature, Vol 504, 12 Dec. 2013). We included their reported change in expression levels for their naïve-like stem cells compared to primed state stem cells. Those values are included in Table 1; FIG. 10.

We discovered that the subset of genes that are differentially expressed in naïve state stem cells compared to primed state stem cells will also be differentially expressed in aggressive or metastatic cancers. Thus agents that target the differentially expressed genes, their gene products or pathways they are involved in will be powerful anti-cancer therapeutics for the treatment or prevention of cancer. For example, referring to Table 1, Column D, the genes NAP1L5, PEGS, USP51, RRAGB, KLRB1 and CCL28, numbers 1-6, all have a 50-fold or higher increase in expression in naïve stem cells over primed stem cells. Agents that reduce their expression, or reduce the amount of the gene product, inhibit the gene product or inhibit the pathways they stimulate will be powerful anti-cancer therapeutics for the treatment or prevention of cancer. Similarly, genes listed as numbers 7-27 all have expression increased by 20-fold or more in naïve stem cells over the parent primed stem cells. Agents that reduce their expression, or reduce the amount of the gene product, inhibit the gene product or inhibit the pathways they stimulate will be powerful anti-cancer therapeutics for the treatment or prevention of cancer. Similarly, genes listed as numbers 28-49 all have expression increased by 10-fold or more in naïve stem cells over the parent primed stem cells. Agents that reduce their expression, or reduce the amount of the gene product, inhibit the gene product or inhibit the pathways they stimulate will be powerful anti-cancer therapeutics for the treatment or prevention of cancer. Genes listed as numbers 50-147 all have expression increased by 4-fold or more in naïve stem cells over the parent primed stem cells. Agents that reduce their expression, or reduce the amount of the gene product, inhibit the gene product or inhibit the pathways they stimulate will be powerful anti-cancer therapeutics for the treatment or prevention of cancer.

Those skilled in the art are familiar with methods and techniques for decreasing expression of a gene or a gene product and for inhibiting pathways the gene products stimulate. One method involves de-stabilizing transcription machinery that assembles at the start site of transcription for a particular gene, including when the transcription machinery is a superenhancer. Another method involves gene therapy methods in which nucleic acids are excised to decrease expression of a gene and/or its gene product. Recent gene editing techniques include but are not limited to CRISPR, Talons, FLPase and cre-LOX. Another method involves administering to the patient an antibody or antibody fragment or derivative that binds to the gene product and inhibits its function, wherein the antibody or antibody derivative can be integrated into a cell, such as an immune cell. Another method involves administering to the patient small molecule that binds to the gene product and inhibits its function. Databases contain information regarding pathways that specific genes or gene products are involved in. Those skilled in the art are familiar with several methods for inhibiting pathways, including by the use of antibodies, small molecules or proteins. For example, IWP2 and Wnt C-59 are small molecules that inhibit Porcupine, which in turn inhibits the Wnt pathway.

Those skilled in the art are familiar with methods for identifying which of the genes with increased expression are the best cancer drug targets. In one aspect of the invention, inhibitory RNAs against each gene are separately tested on stem cells or cancer cells. RNAi's that induce stem cells to differentiate, lose OCT4 expression or inhibit their proliferation are then identified and the gene they target is identified as being a gene or gene product to suppress or inhibit for the treatment or prevention of cancer. RNAi's that induce cancer cells to differentiate or inhibit their proliferation are then identified and the gene they target is identified as being a gene or gene product to suppress or inhibit for the treatment or prevention of cancer. It is important to note that genes that are down-regulated in naïve stem cells could play an even more important role in promoting cancer than the genes that are up-regulated. In the case of genes that are down-regulated, agents that increase their expression, increase the amount of the gene product, are agonists of the gene product or stimulate the pathways the gene products stimulate will be powerful anti-cancer therapeutics for the treatment or prevention of cancer. Genes that are involved in promoting differentiation are preferred. Still referring to Table 1, Column D, genes listed as numbers 1130-1167 all have expression decreased by 100-fold or more in naïve stem cells over the parent primed stem cells. Agents that increase the expression of one or more of these genes, increase the amount of the gene product, are agonists of the gene product or stimulate the pathways the gene products stimulate will be powerful anti-cancer therapeutics for the treatment or prevention of cancer. Genes listed as numbers 1099-1129 all have expression decreased by 50-fold or more in naïve stem cells over the parent primed stem cells. Agents that increase the expression of one or more of these genes, increase the amount of the gene product, are agonists of the gene product or stimulate the pathways the gene products stimulate will be powerful anti-cancer therapeutics for the treatment or prevention of cancer. Genes listed as numbers 1037-1098 all have expression decreased by 20-fold or more in naïve stem cells over the parent primed stem cells. Agents that increase the expression of one or more of these genes, increase the amount of the gene product, are agonists of the gene product or stimulate the pathways the gene products stimulate will be powerful anti-cancer therapeutics for the treatment or prevention of cancer. Genes listed as numbers 980-1036 all have expression decreased by 10-fold or more in naïve stem cells over the parent primed stem cells. Agents that increase the expression of one or more of these genes, increase the amount of the gene product, are agonists of the gene product or stimulate the pathways the gene products stimulate will be powerful anti-cancer therapeutics for the treatment or prevention of cancer. Genes listed as numbers 822-979 all have expression decreased by 4-fold or more in naïve stem cells over the parent primed stem cells. Agents that increase the expression of one or more of these genes, increase the amount of the gene product, are agonists of the gene product or stimulate the pathways the gene products stimulate will be powerful anti-cancer therapeutics for the treatment or prevention of cancer.

Those skilled in the art are familiar with methods and techniques for increasing expression of a gene or a gene product and for stimulating pathways the gene products stimulate. One method involves stabilizing transcription machinery that assembles at the start site of transcription for a particular gene, including when the transcription machinery is a superenhancer. Another method involves gene therapy methods in which nucleic acids are introduced to increase expression of a gene and/or its gene product. Recent gene editing techniques include but are not limited to CRISPR, Talons, FLPase and cre-LOX. Another method involves administering to the patient the gene product, that is to say the protein, itself. Databases contain information regarding pathways that specific genes or gene products are involved in. Those skilled in the art are familiar with several methods for stimulating pathways, including by the use of antibodies, small molecules or proteins. For example CHIR 99021 is a small molecule that inhibits GSK3-b, which in turn stimulates the Wnt pathway.

Those skilled in the art are familiar with methods for identifying which of the genes with decreased expression are the best targets for increasing their expression. In one aspect of the invention, genes involved in differentiation and development are selected as being preferred as anti-cancer drug targets. In one aspect of the invention, cells are separately transfected with nucleic acids encoding the genes whose expression is decreased in naïve stem cells and are also involved in differentiation. They can be transfected into stem cells or cancer cells. In another aspect of the invention, cells are contacted with the gene product or protein itself. Genes or their gene products that induce stem cells or cancer cells to differentiate, lose OCT4 expression or inhibit their proliferation are then identified. An agent to increase expression of the selected gene product, or the protein itself, which may be recombinant, would then be administered to a person diagnosed with cancer or at risk of developing cancer.

Table 2 (FIG. 11) lists the genes that are uniquely altered in NME7-induced naïve state stem cells. These sets of genes that we identified as being differentially expressed in NME7-induced naïve state stem cells can be compared to a sample from a patient to diagnose cancer in a patient, assess its metastatic potential of a patient's cancer, design a treatment for that patient, devise anti-cancer therapeutics that reverse or correct the aberrant gene expression pattern or to discover drugs to treat metastatic cancers, wherein if a subset of these genes are also differentially expressed in the patient, the patient has a cancer, an aggressive cancer or a cancer with a high potential.

Biochemically reverted naïve-like stem cells, such as those described by Theunissen et al and Gafni et al share some naïve state characteristics with our NME7-induced naïve stem cells but fail to satisfy all the criteria of naïve state stem cells. Therefore, they share some of the same changes in gene expression with NME7-induced naïve stem cells. Genes that are differentially expressed only in NME7-induced naïve state stem cells compared to primed state stem cells are listed in Table 2; none of these changes in gene expression has been reported to be indicators of the human naïve state stem cells. In one aspect, genes whose expression is down-regulated make up the set of genes that a patient's sample gene expression is compared to, since many of the down-regulated genes induce differentiation if expressed. In another aspect, genes that have altered expression of 2-fold or more make up the data set that is indicative of cancer or risk of developing cancer or risk of developing metastatic cancer. In another aspect, genes that have altered expression of 4-fold or more make up the data set that is indicative of cancer or risk of developing cancer or risk of developing metastatic cancer. In another aspect, genes that have altered expression of 10-fold or more make up the data set that is indicative of cancer or risk of developing cancer or risk of developing metastatic cancer. In another aspect, genes that have altered expression of 50-fold or more make up the data set that is indicative of cancer or risk of developing cancer or risk of developing metastatic cancer. In another aspect, genes that have altered expression of 100-fold or more make up the data set that is indicative of cancer or risk of developing cancer or risk of developing metastatic cancer. In one aspect of the invention, genes that are uniquely identified in NME7-induced naïve stem cells as having altered expression of 2-fold or more make up the data set that is indicative of cancer or risk of developing cancer or risk of developing metastatic cancer; 4-fold, 10-fold, 50-fold, 100-fold. In another aspect of the invention, genes that have altered expression of 2-fold or more in all three naïve-like stem cells shown in Table 3 (FIG. 12) make up the data set that is indicative of cancer or risk of developing cancer or risk of developing metastatic cancer.

There is another category of genes that could be important targets for anti-cancer drugs. It was recently discovered that in certain cases mega-complexes, called super-enhancers, assemble at a relatively few genes and cause them to be super expressed (Hnisz et al., Cell, 155, 934-947, Nov. 7, 2013). In embryonic stem cells, roughly 40% of all Mediator components pile up at only a few hundred enhancer sites, forming a mega-complex and so are called super-enhancers. Super-enhancers increase expression of the target genes by many times more than typical enhancers so in this way could rapidly execute key cell fate decisions, such as whether to grow pluripotently or differentiate. Bleed through in key cell fate decisions, such as whether to grow pluripotently or differentiate, would have devastating consequences for development of an embryo for example. It is theorized that these genes constitute Master ON/OFF switches that define a de-differentiated stem cell state. There is also some evidence that in cancer cells, super-enhancers assemble at key genes and cause them to be super expressed.

Hnisz et al. devised a method of identifying genes that are regulated by super-enhancers. They identified about 200 genes regulated by super-enhancers in human H1 embryonic primed state stem cells, see Table 4 (FIG. 13). However, our gene expression data of human naïve state stem cells shows that several of the genes listed in Table 4 that are active, occupied by super-enhancers and super expressed in primed state stem cells, are down-regulated in naïve state stem cells (Table 5; FIG. 14) and thus not occupied by super-enhancers. Recall that we showed that the same growth factor, NME7-AB, that drives human primed state stem cells into the earlier naïve state, is the same growth factor that drives regular cancer cells into the metastatic state. That means that the subset of genes that are super-expressed in primed state stem cells but actually have decreased expression in naïve state stem cells is the subset of genes that drives cancer cells to the metastatic state. Therefore, agents that increase the expression of genes listed in Table 5, or their gene products, or the gene products themselves will be powerful anti-cancer and anti-metastasis therapeutics for the treatment or prevention of cancer. Genes listed in Table 5 as numbers 1-27 are not super-expressed as they are in primed state cells (Hnisz et al.). Agents that increase the expression of one or more of these genes, increase the amount of the gene product, are agonists of the gene product or stimulate the pathways the gene products stimulate, or the gene products themselves will be powerful anti-cancer or anti-metastasis therapeutics for the treatment or prevention of cancer. Genes listed in Table 5 as numbers 12-27 have significantly decreased expression compared to primed state cells. Agents that increase the expression of one or more of these genes, increase the amount of the gene product, are agonists of the gene product or stimulate the pathways the gene products stimulate, or the gene products themselves will be powerful anti-cancer or anti-metastasis therapeutics for the treatment or prevention of cancer. In this case, it is desirable to increase expression of these genes or to treat with the gene products themselves. In one aspect of the invention, a nucleic acid including a portion that encodes one of these genes is administered to a person diagnosed with or at risk of developing cancer. In another aspect of the invention, a protein encoded by the gene, which may be derivatized to facilitate entry into a cell, is administered to a person diagnosed with or at risk of developing cancer.

Genes HES3, GNAS, FBXL17, RHOC, VLDLR, GREB1L, EXT1, BRD2 and CDH9 have significantly decreased expression compared to primed state cells and only show significantly decreased expression in NME7-AB induced naïve cells. Agents that increase the expression of one or more of these genes, increase the amount of the gene product, are agonists of the gene product or stimulate the pathways the gene products stimulate, or the gene products themselves will be powerful anti-cancer or anti-metastasis therapeutics for the treatment or prevention of cancer. In this case, it is desirable to increase expression of these genes or to treat with the gene products themselves. In one aspect of the invention, a nucleic acid including a portion that encodes one of these genes is administered to a person diagnosed with or at risk of developing cancer. In another aspect of the invention, a protein encoded by the gene, which may be derivatized to facilitate entry into a cell, is administered to a person diagnosed with or at risk of developing cancer.

Agents that act as described above on genes or their gene products that directly or indirectly promote differentiation are preferred, as cancer cells resemble stem cells as they are de-differentiated. For example, HES3, GNAS, FBXL17, RHOC, VLDLR, GREB1L, EXT1, BRD2 and CDH9 are all super-expressed in primed state stem cells, but all have decreased expression in naïve state stem cells. BRD2 itself regulates expression of 1,450 other genes through its interaction with chromatin. HES3 regulates expression of all basic helix-loop-helix transcription factors. GNAS mediates the activity of a host of factors that are critical for differentiation. None of these three super-enhancer regulated genes was down-regulated in the other naïve-like stem cells. Agents that increase the expression of one or more of these genes, increase the amount of the gene product, are agonists of the gene product or stimulate the pathways the gene products stimulate, or the gene products themselves will be powerful anti-cancer or anti-metastasis therapeutics for the treatment or prevention of cancer.

In one aspect of the invention, the subset of genes regulated by super-enhancers that has altered expression by 2-fold or more in stem cells comprises the data set that is indicative of cancer or risk of developing cancer or risk of developing metastatic cancer.

In a preferred embodiment, genes that could be regulated by super-enhancers but are differentially expressed in naïve state stem cells compared to primed state stem cells comprise the data set that is indicative of cancer or risk of developing cancer or risk of developing metastatic cancer. In a more preferred embodiment, genes that could be regulated by super-enhancers but are down-regulated in naïve state stem cells compared to primed state stem cells comprise the data set that is indicative of cancer or risk of developing cancer or risk of developing metastatic cancer. Many of the genes that exhibit altered expression in our NME7 naïve stem cells, compared to primed state stem cells, are regulated by super-enhancers (Table 5; FIG. 14). One example is the list of super-enhancer regulated genes that has altered expression by 2-fold or more in NME7 naïve stem cells shown in Table 5. Measuring expression levels of super-enhancer regulated genes in a patient sample and determining that their expression levels more closely resemble expression levels in stem cells than in healthy donor samples, is indicative of cancer or risk of developing cancer or risk of developing metastatic cancer. Measuring expression levels of super-enhancer regulated genes in a patient sample and determining that their expression levels more closely resemble expression levels in naïve state stem cells than in healthy donor samples, is indicative of cancer or risk of developing cancer or risk of developing metastatic cancer. Particularly important are the genes that are regulated by super-enhancers that are down-regulated in naive state stem cells, meaning they are turned off. These are genes that induce differentiation. In cancers, they are also turned off or down-regulated as it is known that cancer cell de-differentiate. In another aspect of the invention, the subset of genes regulated by super-enhancers that has decreased expression by 2-fold or more comprises the data set that is indicative of cancer or risk of developing cancer or risk of developing metastatic cancer. One example is the list of super-enhancer regulated genes that has altered decreased expression by 2-fold or more in NME7 naïve stem cells shown in Table 5.

In one aspect of the invention, the subset of genes regulated by super-enhancers that has altered expression by 2-fold or more in naïve stem cells and is indicative of cancer or risk of developing cancer or risk of developing metastatic cancer includes down-regulated BRD2, which itself regulates expression of 1,450 other genes through its interaction with chromatin, down-regulated HES3, which regulates basic helix-loop-helix transcription factors, and down-regulated GNAS, which mediates the activity of a host of factors that are critical for differentiation. None of these three super-enhancer regulated genes was down-regulated in the other naïve-like stem cells. Because these genes are down-regulated in naïve stem cells but are super-enhancer expressed or super expressed in the primed stem cells which have just begun to differentiate, they are key targets for the treatment or prevention of cancer. In this case, it is desirable to increase expression of these genes or to treat with the gene products themselves. In one aspect of the invention, a nucleic acid including a portion that encodes HES3, GNAS, FBXL17, RHOC, VLDLR, GREB1L, EXT1, BRD2 and CDH9 is administered to a person diagnosed with or at risk of developing cancer. In another aspect of the invention, a protein encoded by HES3, GNAS, FBXL17, RHOC, VLDLR, GREB1L, EXT1, BRD2 and CDH9, which may be derivatized to facilitate entry into a cell, is administered to a person diagnosed with or at risk of developing cancer.

In another aspect of the invention, a method for treating a patient with cancer or at risk of developing cancer, involves reversing the altered expression of genes that are regulated by super-enhancers and whose expression is altered by 2-fold or more in naïve state stem cells, including those shown in Table 5. In one case, expression of down-regulated super-enhancer regulated genes is induced. This can be accomplished by inducing the assembly of a super-enhancer or Mediator at the gene whose expression is desired. Alternatively the genes products are introduced. In another case, expression of up-regulated super-enhancer regulated genes is suppressed. This can be accomplished by anti-sense, transcription repressors or by de-stabilization of the super-enhancer or Mediator complex.

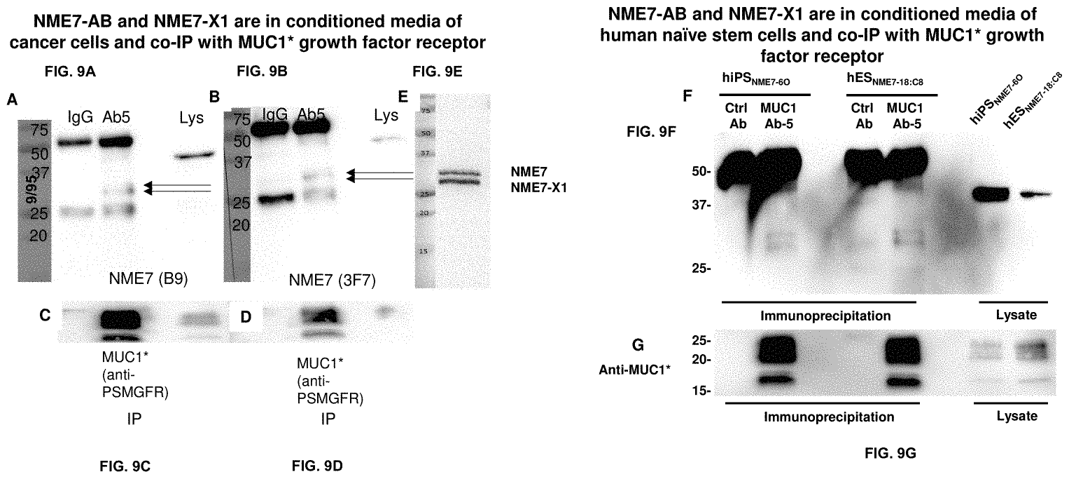

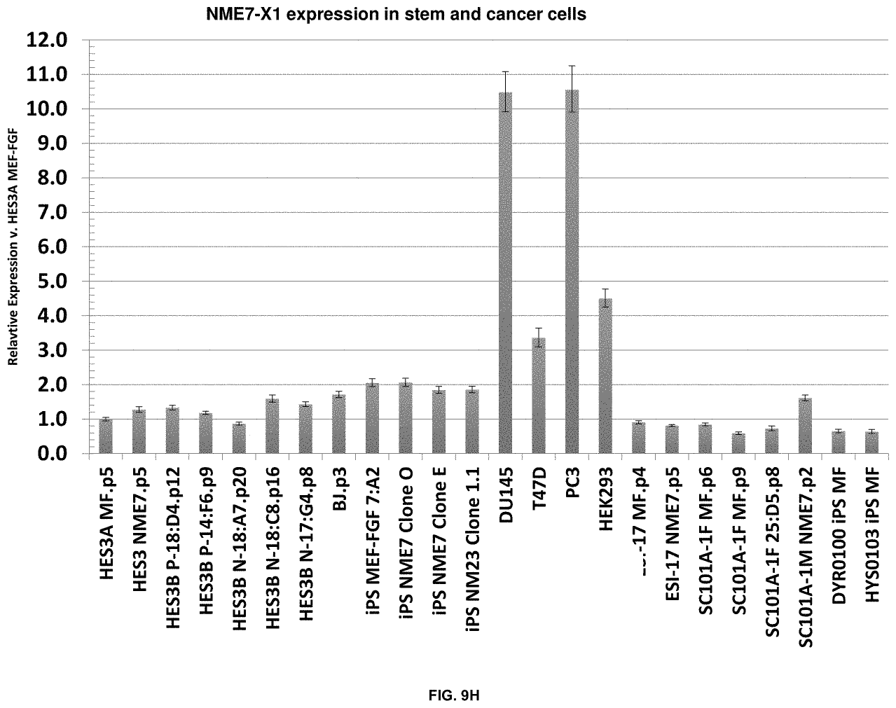

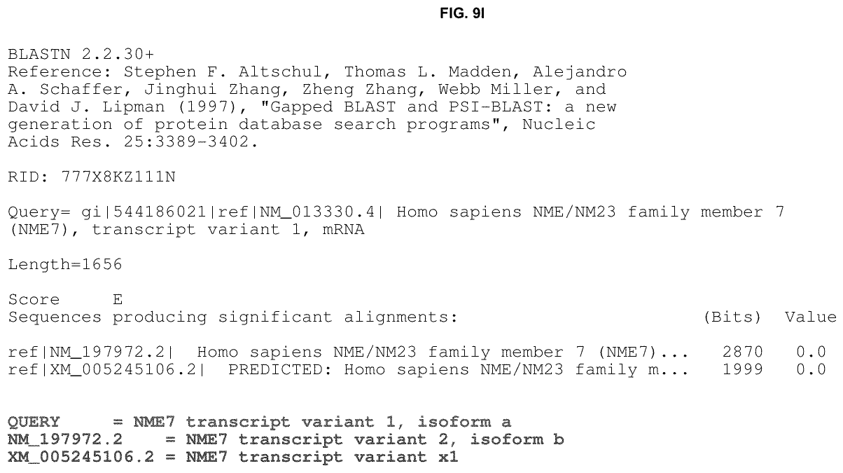

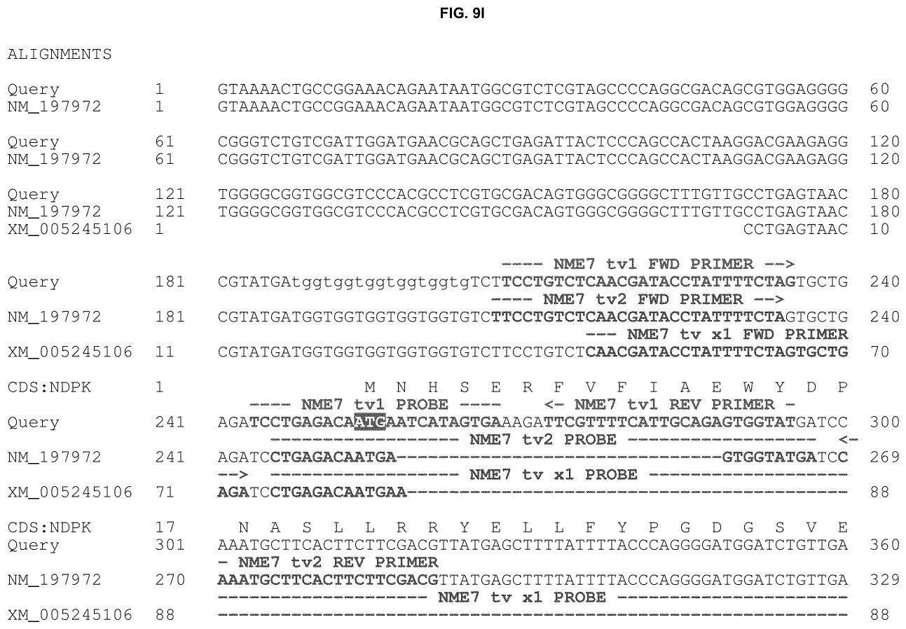

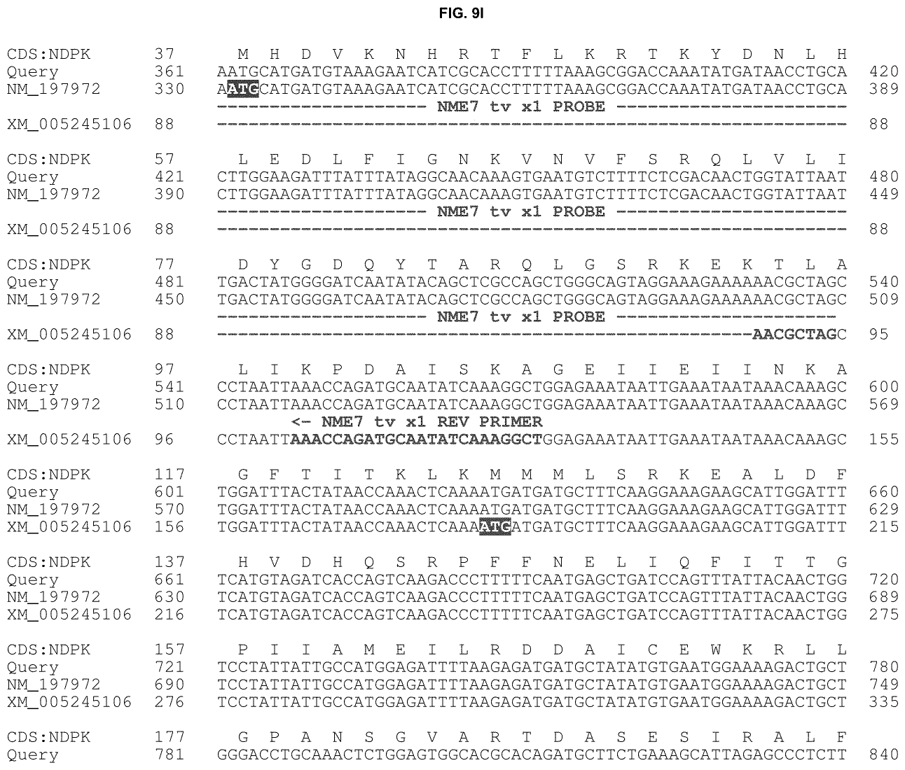

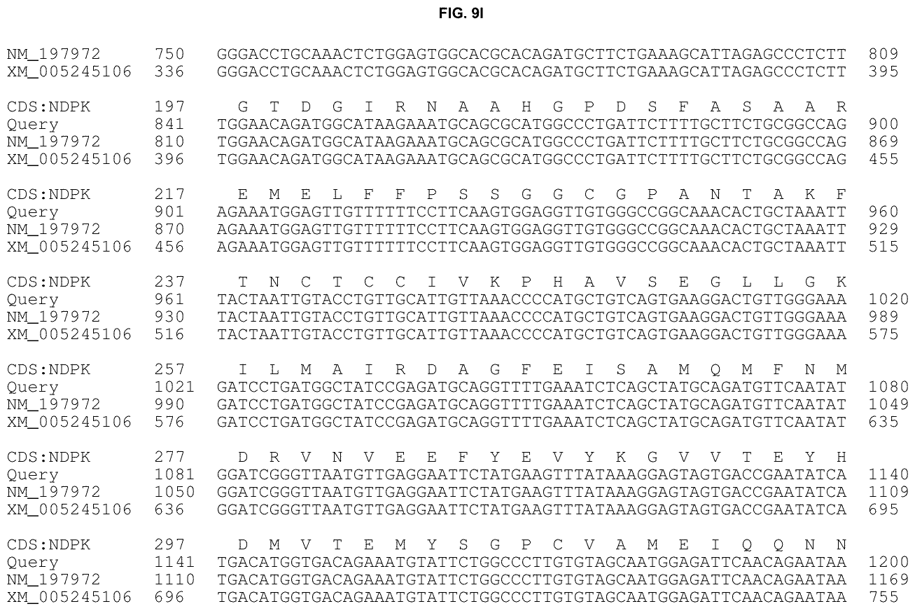

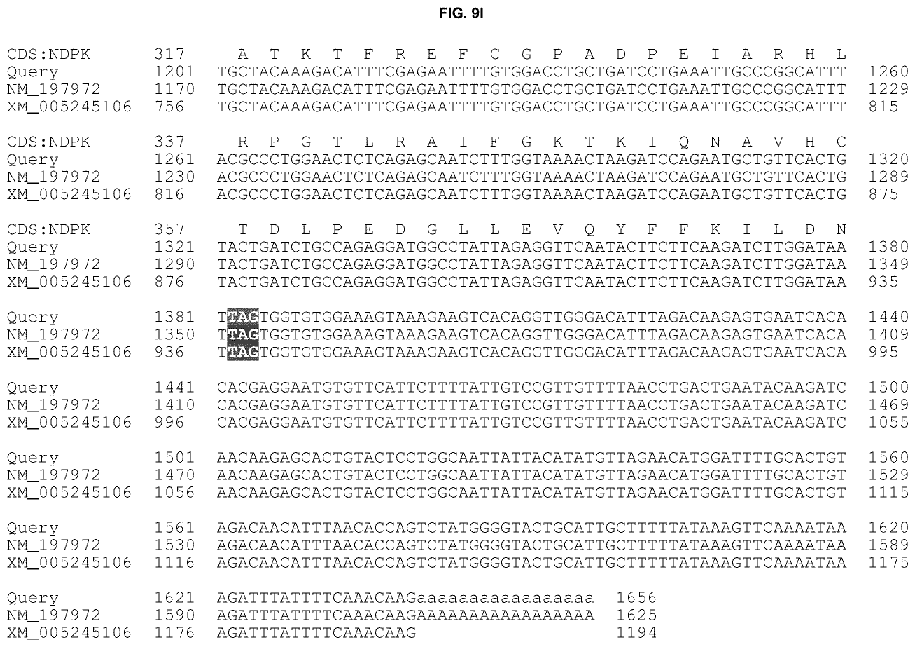

NME7-AB is a naïve stem cell growth factor that binds to MUC1* on stem cells and on cancer cells. Stem cells grown in NME7-AB revert to the earliest naïve state. Cancer cells grown in NME7-AB become more metastatic. Therefore NME7-AB is an important anti-cancer drug target wherein agents that inhibit its actions or inhibit its expression are potent anti-cancer and anti-metastasis therapeutics. Data that emerged from sequencing the human genome frequently lists sequences of what could be alternative splice isoforms for genes. We searched and found the sequence of one such hypothetical alternative splice isoform of NME7. It was listed as NME7-X1, however no evidence or proof of its existence had ever been demonstrated. We made and expressed a recombinant protein from the predicted sequence and designed PCR primers that could detect it in cells and differentiate it from NME7. FIGS. 9A-9G show photographs of Western blots of a co-immunoprecipitation experiment. T47D breast cancer cell extracts were incubated with an antibody against the MUC1 cytoplasmic tail, Ab-5, or a control antibody, IgG, and co-immunoprecipitated. The gels were blotted with two different commercially available anti-NME7 antibodies B9 (A) and CF7 (B). Both gels show unique NME7 bands at ˜33 kDa and ˜30 kDa. The gels were stripped and re-probed with an antibody against the extracellular domain of MUC1*, anti-PSMGFR (C) and (D), which shows that the NME7 species and MUC1* interact. A recombinant NME7-AB and a recombinant NME7-X1 were mixed together and run on a gel, then probed with an anti-NME7 antibody, showing that the two unique NME7 species that are naturally occurring in breast cancer cells and that interact with MUC1* are an NME7-AB-like species and NME7-X1 (E). Western blots of a co-immunoprecipitation experiment. Human induced pluripotent stem, iPS7, or embryonic stem, HES3, cell extracts were incubated with an antibody against the MUC1 cytoplasmic tail, Ab-5, or a control antibody, IgG, and co-immunoprecipitated. The gel was blotted with a commercially available anti-NME7 antibody B9 (F). Both cell types show unique NME7 bands at ˜33 kDa and ˜30 kDa. The gel was stripped and re-probed with an antibody against the extracellular domain of MUC1*, anti-PSMGFR (G), which shows that the NME7 species and MUC1* interact.

FIG. 9H shows a graph of RT-PCR measurement of the expression of NME7-X1 in a panel of human stem cells and cancer cells.

In another approach, a patient sample is tested for expression of NME7-A, NME7-B, NME7 devoid of a DM-10 domain or NME7-X1, wherein the presence of these NME7 variants is an indicator of cancer and levels of NME7 variants is a measure of the metastatic potential of that cancer. In one aspect, the nucleic acids of a patient sample are assayed. In another aspect, an amount of protein is measured in the patient sample. NME7-X1 is overexpressed in cancers such as breast cancer but is grossly overexpressed in prostate cancers. Measuring increased levels of NME7-X1 is an indicator of the presence of or risk of developing cancer. In a preferred embodiment, NME7-X1 is measured and increased expression is an indicator of prostate cancer, risk of developing prostate cancer or is an indicator of the metastatic potential of a cancer. NME7 is normally not expressed at all in adult tissues or is expressed at very low levels. Measuring an increased amount of NME7 or a 30 kDa or 33 kDa NME7 variant is an indicator of the presence of a cancer or high risk of developing a cancer and indicates high metastatic potential of cancer.

Those skilled in the art will be familiar with many techniques that can be used to measure levels of NME variants. Various techniques measure levels of nucleic acids encoding NME7-A, NME7-B or NME7-X1, including PCR, RT-PCR, hybridization assays and sequencing assays. FIG. 9I shows sequence alignment of NME7-A, aka variant 1 or v1, NME7-B, aka variant 2 or v2, and NME7-X1, aka X1. The primer sets listed in Table 6 show primer sequences that will distinguish these NME7 variants from one another. Primer sequences can vary in the length and exact sequences used; the primers listed in Table 6 are meant to be exemplary and not exclusive. Alternatively, nucleic acid hybridization assays can be used to detect NME7 variants and also to distinguish one variant from another. The nucleic acid sequences listed in Table 7 are sequences that will hybridize to some or one NME7 variant but not to another. Nucleic acids can be modified w labels, including optical tags, fluorescent tags, electronic or amplifiable tags.

NME7 and NME7-X1 have a Ca⁺⁺ binding motif that is predicted to bind to nucleic acids. Our studies show that NME7 and NME7-X1 are translocated to the nucleus of stem cells and cancer cells where they regulate transcription of genes that define a cancerous state. A method for treating cancer or reducing the risk of developing cancer involves identifying which genes are regulated by NME7 or NME7-X1 and causing their effect on gene expression to be reversed. Those skilled in the art will be familiar with chromatin immuno precipitation (ChIP and ChIP SEQ) methods in which antibodies that recognize NME7 and NME7-x1 precipitate out nucleic acids to which they are bound. Sequencing then identifies the genes. RT-PCR or RNA SEQ techniques are then employed to determine if binding by NME7 or NME7-X1 induces or suppresses expression of those genes. To treat or prevent cancer, the effects of NME7 or NME7-X1 on the expression of those genes would be reversed. Methods to restore healthy gene expression levels of those genes regulated by NME7 or NME7-X1 are known to those skilled in the art and include gene therapy, gene silencing, inhibitors of the gene products or the gene products themselves.

We have demonstrated that the same growth factor that reverts human stem cells to the naïve state, NME7-AB, is the same growth factor that progresses cancer cells to a more metastatic state. That argues that cancer cells are really like stem cells wherein the most metastatic cancer cells are the most like naïve state stem cells. Therefore, a method for the treatment or prevention of cancer involves inducing cancer cells to differentiate, using methods that stem cells use to differentiate. Stem cells remain pluripotent and cancer cells remain cancerous by suppressing expression of key genes that control differentiation such as those listed in Table 5, and in particular HES3, GNAS, FBXL17, RHOC, VLDLR, GREB1L, EXT1, BRD2 and CDH9. Therefore, a method of treating cancer is to administer to the patient agents that induce stem cells to differentiate. In one aspect of the invention, agents are administered to a patient diagnosed with cancer or at risk of developing cancer that increase the expression of the genes listed in Table 1, Column D that have decreased expression in naïve stem cells. In another aspect of the invention, agents are administered to a patient diagnosed with cancer or at risk of developing cancer that increase the expression of the genes listed in Table 5. In another aspect of the invention, agents are administered to a patient diagnosed with cancer or at risk of developing cancer that increase the expression of HES3, GNAS, FBXL17, RHOC, VLDLR, GREB1L, EXT1, BRD2 and CDH9.

Cancer

There are over 200 types of cancer, of which many may cancer cells express MUC1* aberrantly. General categories of cancer, whether their cancer cells expresses MUC1* aberrantly or not, include the following. This list is not all inclusive and the cancers listed in quotes are the general names of some cancers:

Carcinoma: Cancer that begins in the skin or in tissues that line or cover internal organs and include “skin, lung, colon, pancreatic, ovarian cancers,” epithelial, squamous and basal cell carcinomas, melanomas, papillomas, and adenomas.

Sarcoma: Cancer that begins in bone, cartilage, fat, muscle, blood vessels, or other connective or supportive tissue and include “bone, soft tissue cancers, osteosarcoma, synovial sarcoma, liposarcoma, angiosarcoma, rhabdosarcoma, and fibrosarcoma.

Leukemia: Cancer that starts in blood-forming tissue such as the bone marrow and causes large numbers of abnormal blood cells to be produced and enter the blood and include “leukemia,” lymphoblastic leukemias (ALL and CLL), myelogenous leukemias (AML and CML), T-cell leukemia, and hairy-cell leukemia.

Lymphoma and myeloma: Cancers that begin in the cells of the immune system and include “lymphoma,” T-cell lymphomas, B-cell lymphomas, Hodgkin lymphomas, non-Hodgkin lymphoma, and lymphoproliferative lymphomas.

Central nervous system cancers: Cancers that begin in the tissues of the brain and spinal cord and include “brain and spinal cord tumors,” gliomas, meningiomas, pituitary adenomas, vestibular schwannomas, primary CNS lymphomas, and primitive neuroectodermal tumors.

Not included in the above types listed are metastatic cancers. This is because metastatic cancer cells usually arise from a cell type listed above and the major difference from the above types is that these cells are now present in a tissue from which the cancer cells did not originally develop. Consequently, if the term “metastatic cancer” is used, for accuracy, the tissue from which the cancer cells arose should be included. For “metastatic cancer”, this term is more accurately described as “metastatic (breast, lung, colon, or other type) cancer with spread to the organ in which it has been found.” For example, prostate cancer spreading to bones is stated as metastatic prostate cancer to bone. This is not “bone cancer,” which would be cancer that started in the bone cells.

The present invention may be used to treat any of the above-described types of cancer, preferably those cancer cells that express MUC1* or the truncated form of MUC1, which displays the Primary Sequence of the MUC1 Growth Factor (PSMGFR) region.

Sequence Listing Free Text

As regards the use of nucleotide symbols other than a, g, c, t, they follow the convention set forth in WIPO Standard ST.25, Appendix 2, Table 1, wherein k represents t or g; n represents a, c, t or g; m represents a or c; r represents a or g; s represents c or g; w represents a or t and y represents c or t.

describes NME7 nucleotide sequence (NME7: GENBANK ACCESSION
AB209049)
(SEQ ID NO: 1)
gagatcctgagacaatgaatcatagtgaaagattcgttttcattgcagagtggtatgatc
caaatgcttcacttcttcgacgttatgagcttttattttacccaggggatggatctgttgaaatgca
tgatgtaaagaatcatcgcacctttttaaagcggaccaaatatgataacctgcacttggaagattta
tttataggcaacaaagtgaatgtcttttctcgacaactggtattaattgactatggggatcaatata
cagctcgccagctgggcagtaggaaagaaaaaacgctagccctaattaaaccagatgcaatatcaaa
ggctggagaaataattgaaataataaacaaagctggatttactataaccaaactcaaaatgatgatg
ctttcaaggaaagaagcattggattttcatgtagatcaccagtcaagaccctttttcaatgagctga
tccagtttattacaactggtcctattattgccatggagattttaagagatgatgctatatgtgaatg
gaaaagactgctgggacctgcaaactctggagtggcacgcacagatgcttctgaaagcattagagcc
ctctttggaacagatggcataagaaatgcagcgcatggccctgattcttttgcttctgcggccagag
aaatggagttgttttttccttcaagtggaggttgtgggccggcaaacactgctaaatttactaattg
tacctgttgcattgttaaaccccatgctgtcagtgaaggtatgttgaatacactatattcagtacat
tttgttaataggagagcaatgtttattttcttgatgtactttatgtatagaaaataa.
describes NME7 amino acid sequence (NME7: GENBANK ACCESSION
AB209049)
(SEQ ID NO: 2)
DPETMNHSERFVFIAEWYDPNASLLRRYELLFYPGDGSVEMHDVKNHRTFLKRTKYDNLH
LEDLFIGNKVNVFSRQLVLIDYGDQYTARQLGSRKEKTLALIKPDAISKAGEIIEIINKAGFTITKL
KMMMLSRKEALDFHVDHQSRPFFNELIQFITTGPIIAMEILRDDAICEWKRLLGPANSGVARTDASE
SIRALFGTDGIRNAAHGPDSFASAAREMELFFPSSGGCGPANTAKFTNCTCCIVKPHAVSEGMLNTL
YSVHFVNRRAMFIFLMYFMYRK.
describes NM23-H1 nucleotide sequence (NM23-H1: GENBANK
ACCESSION AF487339)
(SEQ ID NO: 3)
atggtgctactgtctactttagggatcgtctttcaaggcgaggggcctcctatctcaagc
tgtgatacaggaaccatggccaactgtgagcgtaccttcattgcgatcaaaccagatggggtccagc
ggggtcttgtgggagagattatcaagcgttttgagcagaaaggattccgccttgttggtctgaaatt
catgcaagcttccgaagatcttctcaaggaacactacgttgacctgaaggaccgtccattctttgcc
ggcctggtgaaatacatgcactcagggccggtagttgccatggtctgggaggggctgaatgtggtga
agacgggccgagtcatgctcggggagaccaaccctgcagactccaagcctgggaccatccgtggaga
cttctgcatacaagttggcaggaacattatacatggcagtgattctgtggagagtgcagagaaggag
atcggcttgtggtttcaccctgaggaactggtagattacacgagctgtgctcagaactggatctatg
aatga.
NM23-H1 describes amino acid sequence (NM23-H1: GENBANK ACCESSION
AF487339)
(SEQ ID NO: 4)
MVLLSTLGIVFQGEGPPISSCDTGTMANCERTFIAIKPDGVQRGLVGEIIKRFEQKGFRL
VGLKFMQASEDLLKEHYVDLKDRPFFAGLVKYMHSGPVVAMVWEGLNVVKTGRVMLGETNPADSKPG
TIRGDFCIQVGRNIIHGSDSVESAEKEIGLWFHPEELVDYTSCAQNWIYE.
Human NME7-A:
(DNA)
(SEQ ID NO: 5)
atggaaaaaacgctagccctaattaaaccagatgcaatatcaaaggctggagaaataat
tgaaataataaacaaagctggatttactataaccaaactcaaaatgatgatgctttcaaggaaagaa
gcattggattttcatgtagatcaccagtcaagaccctttttcaatgagctgatccagtttattacaa
ctggtcctattattgccatggagattttaagagatgatgctatatgtgaatggaaaagactgctggg
acctgcaaactctggagtggcacgcacagatgcttctgaaagcattagagccctctttggaacagat
ggcataagaaatgcagcgcatggccctgattcttttgcttctgcggccagagaaatggagttgtttt
tttga
(amino acids)
(SEQ ID NO: 6)
MEKTLALIKPDAISKAGEIIEIINKAGFTITKLKMMMLSRKEALDFHVDHQSRPFFNEL
IQFITTGPIIAMEILRDDAICEWKRLLGPANSGVARTDASESIRALFGTDGIRNAAHGPDSFASAAR
EMELFF-
Human NME7-A1:
(DNA)
(SEQ ID NO: 7)
atggaaaaaacgctagccctaattaaaccagatgcaatatcaaaggctggagaaataat
tgaaataataaacaaagctggatttactataaccaaactcaaaatgatgatgctttcaaggaaagaa
gcattggattttcatgtagatcaccagtcaagaccctttttcaatgagctgatccagtttattacaa
ctggtcctattattgccatggagattttaagagatgatgctatatgtgaatggaaaagactgctggg
acctgcaaactctggagtggcacgcacagatgcttctgaaagcattagagccctctttggaacagat
ggcataagaaatgcagcgcatggccctgattcttttgcttctgcggccagagaaatggagttgtttt
ttccttcaagtggaggttgtgggccggcaaacactgctaaatttacttga
(amino acids)
(SEQ ID NO: 8)
MEKTLALIKPDAISKAGEIIEIINKAGFTITKLKMMMLSRKEALDFHVDHQSRPFFNEL
IQFITTGPIIAMEILRDDAICEWKRLLGPANSGVARTDASESIRALFGTDGIRNAAHGPDSFASAAR
EMELFFPSSGGCGPANTAKFT-
Human NME7-A2:
(DNA)
(SEQ ID NO: 9)
atgaatcatagtgaaagattcgttttcattgcagagtggtatgatccaaatgcttcact
tcttcgacgttatgagcttttattttacccaggggatggatctgttgaaatgcatgatgtaaagaat
catcgcacctttttaaagcggaccaaatatgataacctgcacttggaagatttatttataggcaaca
aagtgaatgtcttttctcgacaactggtattaattgactatggggatcaatatacagctcgccagct
gggcagtaggaaagaaaaaacgctagccctaattaaaccagatgcaatatcaaaggctggagaaata
attgaaataataaacaaagctggatttactataaccaaactcaaaatgatgatgctttcaaggaaag
aagcattggattttcatgtagatcaccagtcaagaccctttttcaatgagctgatccagtttattac
aactggtcctattattgccatggagattttaagagatgatgctatatgtgaatggaaaagactgctg
ggacctgcaaactctggagtggcacgcacagatgcttctgaaagcattagagccctctttggaacag
atggcataagaaatgcagcgcatggccctgattcttttgcttctgcggccagagaaatggagttgtt
tttttga
(amino acids)
(SEQ ID NO: 10)
MNHSERFVFIAEWYDPNASLLRRYELLFYPGDGSVEMHDVKNHRTFLKRTKYDNLHLED
LFIGNKVNVFSRQLVLIDYGDQYTARQLGSRKEKTLALIKPDAISKAGEIIEIINKAGFTITKLKMM
MLSRKEALDFHVDHQSRPFFNELIQFITTGPIIAMEILRDDAICEWKRLLGPANSGVARTDASESIR
ALFGTDGIRNAAHGPDSFASAAREMELFF-
Human NME7-A3:
(DNA)
(SEQ ID NO: 11)
atgaatcatagtgaaagattcgttttcattgcagagtggtatgatccaaatgcttcact
tcttcgacgttatgagcttttattttacccaggggatggatctgttgaaatgcatgatgtaaagaat
catcgcacctttttaaagcggaccaaatatgataacctgcacttggaagatttatttataggcaaca
aagtgaatgtcttttctcgacaactggtattaattgactatggggatcaatatacagctcgccagct
gggcagtaggaaagaaaaaacgctagccctaattaaaccagatgcaatatcaaaggctggagaaata
attgaaataataaacaaagctggatttactataaccaaactcaaaatgatgatgctttcaaggaaag
aagcattggattttcatgtagatcaccagtcaagaccctttttcaatgagctgatccagtttattac
aactggtcctattattgccatggagattttaagagatgatgctatatgtgaatggaaaagactgctg
ggacctgcaaactctggagtggcacgcacagatgcttctgaaagcattagagccctctttggaacag
atggcataagaaatgcagcgcatggccctgattcttttgcttctgcggccagagaaatggagttgtt
ttttccttcaagtggaggttgtgggccggcaaacactgctaaatttacttga
(amino acids)
(SEQ ID NO: 12)
MNHSERFVFIAEWYDPNASLLRRYELLFYPGDGSVEMHDVKNHRTFLKRTKYDNLHLED
LFIGNKVNVFSRQLVLIDYGDQYTARQLGSRKEKTLALIKPDAISKAGEIIEIINKAGFTITKLKMM
MLSRKEALDFHVDHQSRPFFNELIQFITTGPIIAMEILRDDAICEWKRLLGPANSGVARTDASESIR
ALFGTDGIRNAAHGPDSFASAAREMELFFPSSGGCGPANTAKFT-
Human NME7-B:
(DNA)
(SEQ ID NO: 13)
atgaattgtacctgttgcattgttaaaccccatgctgtcagtgaaggactgttgggaaa
gatcctgatggctatccgagatgcaggttttgaaatctcagctatgcagatgttcaatatggatcgg
gttaatgttgaggaattctatgaagtttataaaggagtagtgaccgaatatcatgacatggtgacag
aaatgtattctggcccttgtgtagcaatggagattcaacagaataatgctacaaagacatttcgaga
attttgtggacctgctgatcctgaaattgcccggcatttacgccctggaactctcagagcaatcttt
ggtaaaactaagatccagaatgctgttcactgtactgatctgccagaggatggcctattagaggttc
aatacttcttctga
(amino acids)
(SEQ ID NO: 14)
MNCTCCIVKPHAVSEGLLGKILMAIRDAGFEISAMQMFNMDRVNVEEFYEVYKGVVTEY
HDMVTEMYSGPCVAMEIQQNNATKTFREFCGPADPEIARHLRPGILRAIFGKTKIQNAVHCIDLPED
GLLEVQYFF-
Human NME7-B1:
(DNA)
(SEQ ID NO: 15)
atgaattgtacctgttgcattgttaaaccccatgctgtcagtgaaggactgttgggaaa
gatcctgatggctatccgagatgcaggttttgaaatctcagctatgcagatgttcaatatggatcgg
gttaatgttgaggaattctatgaagtttataaaggagtagtgaccgaatatcatgacatggtgacag
aaatgtattctggcccttgtgtagcaatggagattcaacagaataatgctacaaagacatttcgaga
attttgtggacctgctgatcctgaaattgcccggcatttacgccctggaactctcagagcaatcttt
ggtaaaactaagatccagaatgctgttcactgtactgatctgccagaggatggcctattagaggttc
aatacttcttcaagatcttggataattagtga
(amino acids)
(SEQ ID NO: 16)
MNCTCCIVKPHAVSEGLLGKILMAIRDAGFEISAMQMFNMDRVNVEEFYEVYKGVVTEY
HDMVTEMYSGPCVAMEIQQNNATKTFREFCGPADPEIARHLRPGILRAIFGKTKIQNAVHCIDLPED
GLLEVQYFFKILDN-
Human NME7-B2:
(DNA)
(SEQ ID NO: 17)
atgccttcaagtggaggttgtgggccggcaaacactgctaaatttactaattgtacctg
ttgcattgttaaaccccatgctgtcagtgaaggactgttgggaaagatcctgatggctatccgagat
gcaggttttgaaatctcagctatgcagatgttcaatatggatcgggttaatgttgaggaattctatg
aagtttataaaggagtagtgaccgaatatcatgacatggtgacagaaatgtattctggcccttgtgt
agcaatggagattcaacagaataatgctacaaagacatttcgagaattttgtggacctgctgatcct
gaaattgcccggcatttacgccctggaactctcagagcaatctttggtaaaactaagatccagaatg
ctgttcactgtactgatctgccagaggatggcctattagaggttcaatacttcttctga
(amino acids)
(SEQ ID NO: 18)
MPSSGGCGPANTAKFTNCTCCIVKPHAVSEGLLGKILMAIRDAGFEISAMQMFNMDRVN
VEEFYEVYKGVVTEYHDMVTEMYSGPCVAMEIQQNNATKTFREFCGPADPEIARHLRPGTLRAIFGK
TKIQNAVHCTDLPEDGLLEVQYFF-
Human NME7-B3:
(DNA)
(SEQ ID NO: 19)
atgccttcaagtggaggttgtgggccggcaaacactgctaaatttactaattgtacctg
ttgcattgttaaaccccatgctgtcagtgaaggactgttgggaaagatcctgatggctatccgagat
gcaggttttgaaatctcagctatgcagatgttcaatatggatcgggttaatgttgaggaattctatg
aagtttataaaggagtagtgaccgaatatcatgacatggtgacagaaatgtattctggcccttgtgt
agcaatggagattcaacagaataatgctacaaagacatttcgagaattttgtggacctgctgatcct
gaaattgcccggcatttacgccctggaactctcagagcaatctttggtaaaactaagatccagaatg
ctgttcactgtactgatctgccagaggatggcctattagaggttcaatacttcttcaagatcttgga
taattagtga
(amino acids)
(SEQ ID NO: 20)
MPSSGGCGPANTAKFTNCTCCIVKPHAVSEGLLGKILMAIRDAGFEISAMQMFNMDRVN
VEEFYEVYKGVVTEYHDMVTEMYSGPCVAMEIQQNNATKTFREFCGPADPEIARHLRPGTLRAIFGK
TKIQNAVHCTDLPEDGLLEVQYFFKILDN--
Human NME7-AB:
(DNA)
(SEQ ID NO: 21)
atggaaaaaacgctagccctaattaaaccagatgcaatatcaaaggctggagaaataat
tgaaataataaacaaagctggatttactataaccaaactcaaaatgatgatgctttcaaggaaagaa
gcattggattttcatgtagatcaccagtcaagaccctttttcaatgagctgatccagtttattacaa
ctggtcctattattgccatggagattttaagagatgatgctatatgtgaatggaaaagactgctggg
acctgcaaactctggagtggcacgcacagatgcttctgaaagcattagagccctctttggaacagat
ggcataagaaatgcagcgcatggccctgattcttttgcttctgcggccagagaaatggagttgtttt
ttccttcaagtggaggttgtgggccggcaaacactgctaaatttactaattgtacctgttgcattgt
taaaccccatgctgtcagtgaaggactgttgggaaagatcctgatggctatccgagatgcaggtttt
gaaatctcagctatgcagatgttcaatatggatcgggttaatgttgaggaattctatgaagtttata
aaggagtagtgaccgaatatcatgacatggtgacagaaatgtattctggcccttgtgtagcaatgga
gattcaacagaataatgctacaaagacatttcgagaattttgtggacctgctgatcctgaaattgcc
cggcatttacgccctggaactctcagagcaatctttggtaaaactaagatccagaatgctgttcact
gtactgatctgccagaggatggcctattagaggttcaatacttcttcaagatcttggataattagtg
a
(amino acids)
(SEQ ID NO: 22)
MEKTLALIKPDAISKAGEIIEIINKAGFTITKLKMMMLSRKEALDFHVDHQSRPFFNEL
IQFITTGPIIAMEILRDDAICEWKRLLGPANSGVARTDASESIRALFGIDGIRNAAHGPDSFASAAR
EMELFFPSSGGCGPANTAKFTNCTCCIVKPHAVSEGLLGKILMAIRDAGFEISAMQMFNMDRVNVEE
FYEVYKGVVTEYHDMVTEMYSGPCVAMEIQQNNATKTFREFCGPADPEIARHLRPGTLRAIFGKTKI
QNAVHCTDLPEDGLLEVQYFFKILDN--
Human NME7-AB1:
(DNA)
(SEQ ID NO: 23)
atggaaaaaacgctagccctaattaaaccagatgcaatatcaaaggctggagaaataat
tgaaataataaacaaagctggatttactataaccaaactcaaaatgatgatgctttcaaggaaagaa
gcattggattttcatgtagatcaccagtcaagaccctttttcaatgagctgatccagtttattacaa
ctggtcctattattgccatggagattttaagagatgatgctatatgtgaatggaaaagactgctggg
acctgcaaactctggagtggcacgcacagatgcttctgaaagcattagagccctctttggaacagat
ggcataagaaatgcagcgcatggccctgattcttttgcttctgcggccagagaaatggagttgtttt
ttccttcaagtggaggttgtgggccggcaaacactgctaaatttactaattgtacctgttgcattgt
taaaccccatgctgtcagtgaaggactgttgggaaagatcctgatggctatccgagatgcaggtttt
gaaatctcagctatgcagatgttcaatatggatcgggttaatgttgaggaattctatgaagtttata
aaggagtagtgaccgaatatcatgacatggtgacagaaatgtattctggcccttgtgtagcaatgga
gattcaacagaataatgctacaaagacatttcgagaattttgtggacctgctgatcctgaaattgcc
cggcatttacgccctggaactctcagagcaatctttggtaaaactaagatccagaatgctgttcact
gtactgatctgccagaggatggcctattagaggttcaatacttcttctga
(amino acids)
(SEQ ID NO: 24)
MEKTLALIKPDAISKAGEIIEIINKAGFTITKLKMMMLSRKEALDFHVDHQSRPFFNEL
IQFITTGPIIAMEILRDDAICEWKRLLGPANSGVARTDASESIRALFGTDGIRNAAHGPDSFASAAR
EMELFFPSSGGCGPANTAKFTNCTCCIVKPHAVSEGLLGKILMAIRDAGFEISAMQMFNMDRVNVEE
FYEVYKGVVTEYHDMVTEMYSGPCVAMEIQQNNATKTFREFCGPADPEIARHLRPGTLRAIFGKTKI
QNAVHCTDLPEDGLLEVQYFF-
Human NME7-X1
(DNA)
(SEQ ID NO: 25)
atgatgatgctttcaaggaaagaagcattggattttcatgtagatcaccagtcaagacc
ctttttcaatgagctgatccagtttattacaactggtcctattattgccatggagattttaagagat
gatgctatatgtgaatggaaaagactgctgggacctgcaaactctggagtggcacgcacagatgctt
ctgaaagcattagagccctctttggaacagatggcataagaaatgcagcgcatggccctgattcttt
tgcttctgcggccagagaaatggagttgttttttccttcaagtggaggttgtgggccggcaaacact
gctaaatttactaattgtacctgttgcattgttaaaccccatgctgtcagtgaaggactgttgggaa
agatcctgatggctatccgagatgcaggttttgaaatctcagctatgcagatgttcaatatggatcg
ggttaatgttgaggaattctatgaagtttataaaggagtagtgaccgaatatcatgacatggtgaca
gaaatgtattctggcccttgtgtagcaatggagattcaacagaataatgctacaaagacatttcgag
aattttgtggacctgctgatcctgaaattgcccggcatttacgccctggaactctcagagcaatctt
tggtaaaactaagatccagaatgctgttcactgtactgatctgccagaggatggcctattagaggtt
caatacttcttcaagatcttggataattag
(amino acids)
(SEQ ID NO: 26)
MMMLSRKEALDFHVDHQSRPFFNELIQFITTGPIIAMEILRDDAICEWKRLLGPANSGV
ARTDASESIRALFGTDGIRNAAHGPDSFASAAREMELFFPSSGGCGPANTAKFTNCTCCIVKPHAVS
EGLLGKILMAIRDAGFEISAMQMFNMDRVNVEEFYEVYKGVVTEYHDMVTEMYSGPCVAMEIQQNNA
TKTFREFCGPADPEIARHLRPGTLRAIFGKTKIQNAVHCTDLPEDGLLEVQYFFKILDN*

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following

All of the references cited herein are incorporated by reference in their entirety.

Those skilled in the art will recognize, or be able to ascertain using no more than routine experimentation, many equivalents to the specific embodiments of the invention specifically described herein.

1. A method of treating cancer comprising comparing whether any of about 3 to 17 genes of transcriptome signature of NME induced naïve state stem cell, whether expressed or inhibited, are also expressed or inhibited in the cancer cell, identifying gene that is expressed or inhibited and turning the gene on or off or treating with the gene product rather than turning the gene on.

2. The method according to 1, wherein if turning gene off is desired, comprising disrupting the Super Mediator complex that super enhances the gene.

3. The method according to 1, wherein if turning gene on is desired, inducing Superenhancer complex that super enhances the gene to bind to the gene.

4. The method according to 3, wherein the gene is HES3, GNAS, FBXL17, RHOC, VLDLR, GREB1L, EXT1, BRD2 and CDH9.

5. The method according to claim 1, wherein NME induced naïve state stem cell transcriptome signature is represented in Tables 1, 2, 3, and 5.

6. The method according to 1, wherein if turning gene on is desired, the proteins themselves are administered to the patient.

7. The method according to 6, wherein the proteins are HES3, GNAS, FBXL17, RHOC, VLDLR, GREB1L, EXT1, BRD2 and CDH9.

8. A method of changing a cancer cell to normal cell comprising turning on any of the genes or any combination thereof in Table 5.

9. The method according to claim 8, wherein the cancer cell is in a patient, and further the method comprises administering to a patient a compound that causes expression of any of the genes or any combination thereof in Table 5.
